(19) United States
(12) Patent Application Publication
(10) Pub. No.: US 2010/0143393 A1
(43) Pub. Date: Jun. 10, 2010

(54) NOVEL INFLUENZA M2 VACCINES

(76) Inventors: Gale Smith, Rockville, MD (US); Rick Bright, Washington, DC (US); D. Craig Wright, Rockville, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/300,427

(22) PCT Filed: May 11, 2007
(86) PCT No.: PCT/US07/11373

§ 371 Date: Feb. 12, 2010

Related U.S. Application Data
(60) Provisional application No. 60/799,343, filed on May 11, 2006.

(51) Int. Cl. A61K 39/145; C07K 14/00; A61P 31/16

(52) U.S. Cl. 424/186.1; 530/350

(57) ABSTRACT

The present invention includes novel influenza antigenic formulations and vaccines that comprise influenza M2 peptide and VLPs comprising influenza M2 protein. The invention also includes methods of making and administering the novel antigenic formulation and vaccine. The invention also include methods of inducing immunity to ameliorate and/or prevent influenza infections in a subject.

This application claims priority to provisional application 60/799,343, filed, May 11, 2006, which is incorporated by reference herein in its entirety for all purposes.

### BACKGROUND

Influenza virus is a member of the Orthomyxoviridae family (for review, see Murphy and Webster, 1996). There are three subtypes of influenza viruses designated A, B, and C that infect humans. The influenza virion contains a segmented negative-sense RNA genome. The influenza virion includes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (PA), and nonstructural protein 2 (NS2) proteins. The HA, NA, M1, and M2 are membrane associated, whereas NP, PB1, PB2, PA, and NS2 are nucleocapsid associated proteins. The NS1 is the only nonstructural protein not associated with virion particles but specific for influenza-infected cells. The M1 protein is the most abundant protein in influenza particles. The HA and NA proteins are envelope glycoproteins, responsible for virus attachment and penetration of the viral particles into the cell, and are the major immunodominant epitopes for virus neutralization and protective immunity. Both HA and NA proteins are considered the most important components for prophylactic influenza vaccines because they are highly immunogenic. However, these proteins can, and often do, change from strain to strain. Due to the variability of these two proteins, a broad spectrum, long lasting vaccine against influenza A has so far not been developed. The influenza vaccine commonly used, has to be adapted almost every year to follow the antigenic drift of the virus. When more drastic changes occur in the virus, known as an antigenic shift, the vaccine is no longer protective.

The M2 protein of influenza A also has been shown to have immunogenic activity. A synthetic peptide containing a N terminus 24 amino acid sequence of M2 coupled to either KLH or OMPC has been shown by others to be immunogenic in multiple animal species, including non-human primates. Passive transfer of hyperimmune primate antisera to mice led to protection when challenged only with a virulent seasonal Influenza A isolate. These synthetic peptide conjugate vaccines however do not generate protective responses against potentially pandemic Influenza A H5N1 isolates. Thus, there is a need for a vaccine capable of inducing broader, more cross-reactive immunity to type A influenza viruses.

### SUMMARY OF THE INVENTION

The invention includes a method for the prevention or amelioration of influenza virus infection in a subject, comprising administering to said subject an M2 peptide or fragments thereof. In one embodiment, said M2 peptide is formulated with Novasomes. In another embodiment, said fragment comprises a peptide from the group consisting of MSLLTEVET, MSLLTEVETP, MSLLTEVETC and MSLLTEVETPC.

The invention also comprises an antigenic formulation or vaccine comprising a M2 peptide or fragments thereof. In one embodiment, said fragment comprises a peptide selected from the group consisting of MSLLTEVET, MSLLTEVETP, MSLLTEVETC and MSLLTEVETPC.

The invention also comprises a chimeric M2-M1 protein, wherein a M2 peptide fragment is fused at or near the N-terminus of the M1 protein. In one embodiment, said chimeric construct comprises the M2 fragment which consists of PIRNEWGCRCNGSSD.

The invention also comprises a VLP comprising a M2-M1 chimera.

The invention also comprises a VLP comprising a M2-HA and/or M2-NA chimera.

The invention also comprises a method for the prevention or amelioration of influenza virus infection of a subject, comprising administering to said subject a VLP comprising a M2-M1 chimera.

### BRIEF DESCRIPTION OF THE DRAWINGS

FIG. 1 illustrates the insertion of the M2 peptide into the M1 protein. O depicts the outer domain, H depicts the helix domain and I depicts the internal domain.

FIG. 2 illustrates the prediction of the outer, helix and inside portions of the M2 protein. O depicts the outer domain, H depicts the helix domain and I depicts the internal domain.

FIG. 3 illustrates the prediction of the outer, helix and inside portions of the M1 protein. O depicts the outer domain, H depicts the helix domain and I depicts the internal domain.

FIG. 4 illustrates an alignment of M2 proteins from different influenza viruses.

FIG. 5 illustrates the alignment of M1 proteins from difference influenza viruses

### DETAILED DESCRIPTION

As used herein the term “adjuvant” refers to a compound that, when used in combination with a specific immunogen in a formulation, augments or otherwise alters or modifies the resultant immune response. Modification of the immune response includes intensification or broadening the specificity of either or both antibody and cellular immune responses. Modification of the immune response can also mean decreasing or suppressing certain antigen-specific immune responses.

As used herein the term “avian influenza virus” refers to influenza viruses found chiefly in birds but that can also infect humans or other animals. In some instances, avian influenza viruses may be transmitted or spread from one human to another. An avian influenza virus that infects humans has the potential to cause an influenza pandemic, i.e., morbidity and/or mortality in humans. A pandemic occurs when a new strain of influenza virus (a virus to which humans have no natural immunity) emerges, spreading beyond individual localities, possibly around the globe, and infecting many humans at once.

As use herein, the term “antigenic formulation” or “antigenic composition” refers to a preparation which, when administered to a subject will induce an immune response. Said immune response may include an induction of specific antibodies that may be used for diagnostic and/or therapeutic purposes.

As used herein, the term “chimeric protein” refers to a fusion protein between two heterologous proteins. Heterologous proteins are proteins from different organisms, including different antigenic variations of the same organism. Examples of chimeric proteins are exemplified below, but comprise M2-M1, M2-HA and/or M2-NA.

As used herein the term “seasonal influenza virus” refers to the influenza viral strains that have been determined to be passing within the human population for a given influenza season based on epidemiological surveys conducted by National Influenza Centers worldwide. These epidemiological studies, and some isolated influenza viruses, are sent to one of four World Health Organization (WHO) reference laboratories, one of which is at the Centers for Disease Control and Prevention (CDC) in Atlanta for detailed testing. These laboratories test how well antibodies made to the current vaccine react to the circulating virus and new flu viruses. This information, along with information about flu activity, is summarized and presented to an advisory committee of the U.S. Food and Drug Administration (FDA) and at a WHO meeting. These meetings result in the selection of three viruses (two subtypes of influenza A viruses and one influenza B virus) to go into flu vaccines for the following fall and winter. The selection occurs in February for the northern hemisphere and in September for the southern hemisphere. Usually, one or two of the three virus strains in the vaccine changes each year.

As use herein, the term “subject” or “patient” refers to, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered.

As used herein, the term “vaccine” refers to a formulation which contains peptides, chimeric proteins and/or VLPs of the present invention, which is in a form that is capable of being administered to a subject and which induces an immune response sufficient to induce immunity to prevent and/or ameliorate an infection and/or to reduce at least one symptom of an infection and/or to enhance the efficacy of another dose said peptides, chimeric proteins and/or VLPs of the present invention or other vaccine. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

### Peptides, Chimeric Proteins and VLPs of the Invention

Influenza virus undergoes frequent and unpredictable changes; therefore, after natural infection, the effective period of protection provided by the host's immunity may only be a few years against the new strains of virus circulating in the community. Thus, there is a need for a vaccine capable of inducing broader, more cross-reactive immunity to type A influenza viruses.

One such component may be M2, a structurally conserved influenza A viral surface protein. M2 mRNA is encoded by RNA segment 7 of the influenza A virus. It is encoded by a spliced mRNA (Lamb et al., (1981) PNAS 4170-4174). Like the hemagglutinin and the neuraminidase, the M2 protein is an integral membrane protein of the influenza A virus. However, the protein is much smaller, only 97 amino acids long. 24 amino acids at the amino terminus are exposed outside the membrane surface (O), 19 amino acids span the lipid bilayer (H), while the remaining 54 residues are located on the cytoplasmic side of the membrane (I) (Lamb et al. (1985) Cell 40, 627 to 633.).

The M2 protein is abundantly expressed at the cell surface of influenza A infected cells (Lamb et al. (1985) Cell, 40, 627 to 633). The protein is also found in the membrane of the virus particle itself, but in much smaller quantities, 14 to 68 molecules of M2 per virion (Zebedee and Lamb (1988) J. Virol. 62, 2762 to 72). The M2 protein is posttranslationally modified by the addition of a palmitic acid on cysteine at position 50 (Sugrue et al. (1990) Virology 179, 51 to 56).

The M2 protein is a homotetramer composed of two disulfide-linked dimers, which are held together by noncovalent interactions (Sugrue and Hay (1991) Virology 180, 617 to 624). By site-directed mutagenesis, Holsinger and Lamb, (1991) Virology 183, 32 to 43, demonstrated that the cysteine residues at positions 17 and 19 are involved in disulfide bridge formation. Only the cysteine at position 17 is present in all viruses analyzed. In the virus strains where cysteine 19 is also present, it is not known whether a second disulfide bridge is formed in the same dimer (already linked by Cys 17-Cys 17) or with the other dimer.

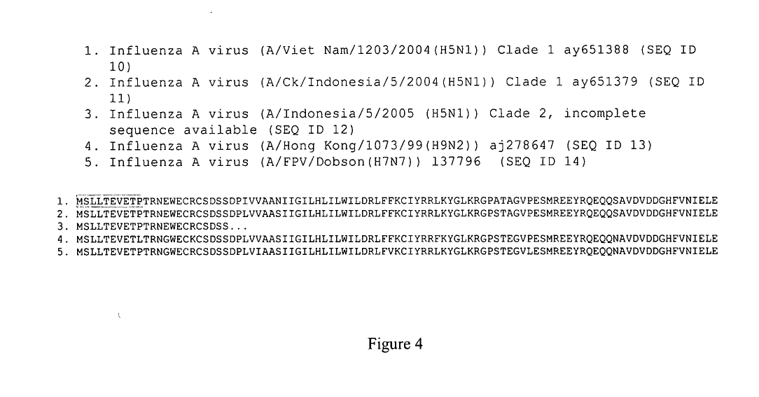

M2 protein is highly conserved among influenza A virus (see FIG. 4). Successful vaccination with a M2-based vaccine may induce a protective immune response against multiple strains of influenza A virus, including avian influenza viruses with pandemic potential.

Thus, the invention comprises a method for the prevention, amelioration and/or treatment of influenza virus infection in a subject, comprising administering to said subject an M2 peptide, or fragments thereof. In one embodiment, said fragments comprise two small conserved N terminus peptide sequences which were found in both seasonal and avian influenza viral isolates, MSLLTEVET (SEQ ID NO. 1) and MSLLTEVETP (SEQ ID NO. 2). In another embodiment, said fragments consists of MSLLTEVET and MSLLTEVETP. In another embodiment, said fragments consists essentially of MSLLTEVET and MSLLTEVETP. In another embodiment, said fragments comprise a peptide selected from the group consisting of MSLLTEVETC (SEQ ID NO. 3) and MSLLTEVETPC (SEQ ID NO. 4). These two peptides have a cysteine at the C-terminus in order to couple said peptides to another agent using the thiol group. In another embodiment, said peptide has a cysteine at the N-terminus (CMSLLTEVET (SEQ ID NO. 6) and CMSLLTEVETP (SEQ ID NO. 7)). In another embodiment, said peptides are formulated with an adjuvant.

The invention also comprises M2-M1 chimeric proteins wherein said chimeric proteins comprises a M2, or fragments thereof, fused to the N-terminus of a M1 protein. Said fusion protein can be administered to a subject to induce an immune response in a subject to prevent, ameliorate and/or treat an influenza virus infection. Thus, the invention comprises a chimeric M2-M1 construct, wherein a M2 peptide fragment is fused at or near the N-terminus of the M1 protein. In another embodiment, said M2 fragment consists of PIRNEWGCRCNGSSD (SEQ ID NO. 5). In another embodiment, said M2 fragment comprises PIRNEWGCRCNGSSD. In another embodiment, said fragment is inserted between about residues 9 and 10 of said M1 protein. In another embodiment, said M2 and/or M1 protein is derived from an avian influenza virus. In another embodiment, said M2 and/or M1 protein is derived from a seasonal influenza virus. In one embodiment, said M2 is derived from a seasonal influenza virus as said virus M1 is derived from an avian influenza virus such as a M1 from a H5N1 strain. In another embodiment, said M2 seasonal/M1 avian chimera is incorporated into a VLP.

Peptides in accordance with the invention can be prepared synthetically, by recombinant DNA technology or chemical synthesis. Peptide epitopes may be synthesized individually or as polyepitopic peptides. Although the peptide will preferably be substantially free of other naturally occurring host cell proteins and fragments thereof, in some embodiments the peptides may be synthetically conjugated to native fragments or particles.

The peptides of the invention can be prepared in a wide variety of ways. For the preferred relatively short size, the peptides can be synthesized in solution or on a solid support in accordance with conventional techniques. Various automatic synthesizers are commercially available and can be used in accordance with known protocols (for example, Stewart & Young, SOLID PHASE PEPTIDE SYNTHESIS, 2D. ED., Pierce Chemical Co., 1984). Further, individual peptide epitopes can be joined using chemical ligation to produce larger peptides that are still within the bounds of the invention.

The nucleotide coding sequence for peptides of the preferred lengths contemplated herein can be synthesized by chemical techniques, for example, the phosphotriester method of Matteucci, et al. (1981) J. Am. Chem. Soc. 103:3185. Peptide analogs can be made simply by substituting the appropriate and desired nucleic acid base(s) for those that encode the native peptide sequence; exemplary nucleic acid substitutions are those that encode an amino acid defined by the motifs/supermotifs herein. The coding sequence can then be provided with appropriate linkers and ligated into expression vectors commonly available in the art. These vectors can be transformed into suitable hosts to produce the desired peptide and/or chimeric protein. A number of such vectors and suitable host systems are known in the art. For expression of a peptide and/or chimeric protein of the invention, the coding sequence can be cloned into a vector comprising an operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence (see below for examples). The resulting expression vectors are transformed into suitable host cells. In one embodiment, said nucleic acids encoding for the peptides of the invention, M2-M1, M2-HA, M2-NA and/or M1 protein can be constructed and used to transfect, infect, or transform a suitable host cell with said expression vector, e.g., a baculovirus. The host cell is cultured under conditions that permit the expression of said peptides and/or chimeric proteins or permit the formation of VLPs of the invention (see below).

The invention also provides a method for producing VLPs derived from a recombinant construct that encodes said M2-M1 chimera, M2-HA, M2-NA and/or an influenza M1 protein in a host cell.

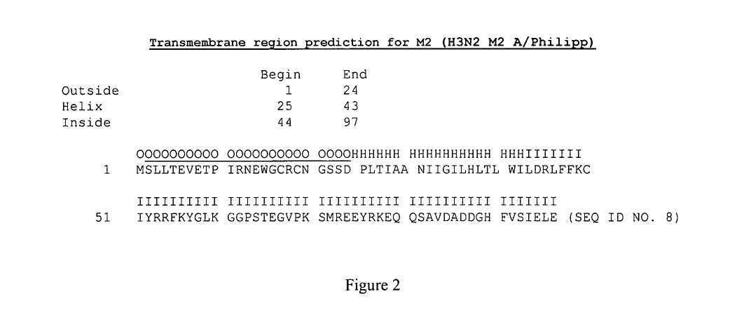

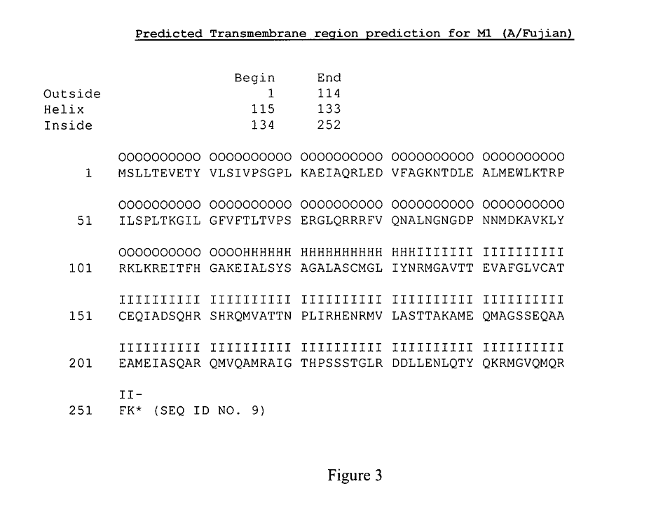

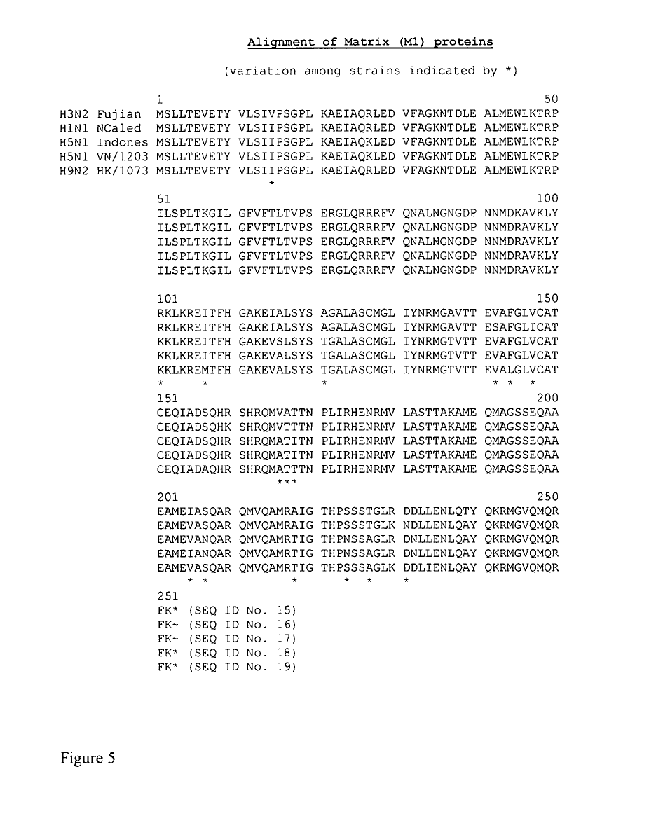

In general, virus-like particles lack a viral genome and, therefore, are noninfectious. In addition, virus-like particles can often be produced in large quantities by heterologous expression and can be easily purified. Virus-like particles (“VLPs”) comprises at least a viral core protein. This core protein will drive budding and release of particles from a host cell. Examples of such proteins comprise RSV M, influenza M1, HIV gag, and vesicular stomatis, Newcastle virus M, virus (VSV) M protein, any of which may be used to produce VLPs (see copending applications U.S. 60/901,652, filed Feb. 16, 2007, and PCT/US2006/030319, filed Aug. 3, 2006, incorporated by reference herein in their entireties for all purposes). As an example, the M1 from an influenza virus and/or the chimeric M2-M1 chimeric molecule will drive VLP formation resulting in a VLP comprising a complete M2 extracellular domain. The M2-M1 chimeric protein will likely have the complete M2 extracellular domain (˜24 amino acids) exposed on the surface of VLPs because predictions show the N-terminal parts of both M1 and M2 are exposed on the outer surface (see predictions, FIGS. 2 and 3). M1, also conserved among influenza A viruses (FIG. 5), may be important vaccine component. The invention also comprises a VLP comprising the chimeric M2-M1 protein. In one embodiment, said VLP also comprises an intact M1 protein. In another embodiment, said VLP comprises an influenza M1 and a chimeric M2, wherein said chimeric M2 comprises a portion of influenza HA and/or NA protein. In another embodiment, said chimeric M2 comprising the transmembrane and/or C-terminal domain of influenza HA or NA is fused to the external domain of M2. In another embodiment, said chimeric M2 has the natural transmembrane and/or C-terminal domain of M2 replaced with the transmembrane and/or C-terminal domain of influenza HA and/or NA.

The invention also comprises variants of the said peptides and chimeric proteins. The variants may contain alterations in the amino acid sequences of the constituent proteins. The term “variant” with respect to a protein refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating the peptides and chimeric proteins of the invention. Thus, the invention also encompasses using known methods of peptide engineering and recombinant DNA technology to improve or alter the characteristics of the peptides and chimeric proteins of the invention.

The invention further comprises peptide variants and chimeric proteins variants which show substantial biological activity, e.g., able to elicit an effective antibody response when administered to a subject.

Methods of cloning said influenza M1, peptides and chimeric proteins of the invention are known in the art. For example, the gene encoding M2 protein, or fragments thereof, can be chemically synthesized as a synthetic gene or can be isolated by RT-PCR from polyadenylated mRNA extracted from cells which had been infected with the said virus. The resulting gene product can be cloned as a DNA insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids or bacmids.

Thus, the invention comprises nucleotides that encode the peptides and chimeric proteins alone or cloned into an expression vector that can be expressed in a cell. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In one embodiment, said nucleotides encode for a peptide and/or said chimeric protein. In another embodiment, the expression vector is a baculovirus vector.

In some embodiments, mutations containing alterations which produce silent substitutions, additions, or deletions, but do not alter the properties or activities of the encoded peptides or chimeric proteins or how they are made. Nucleotide variants can be produced for a variety of reasons, e.g., to optimize codon expression for a particular host. See U.S. patent publication 2005/0118191, herein incorporated by reference in its entirety for all purposes.

In addition, the nucleotides can be sequenced to ensure that the correct coding regions were cloned and do not contain any unwanted mutations. The nucleotides can be subcloned into an expression vector (e.g. baculovirus) for expression in any cell. The above is only one example of how the M1, M2, peptides and/or chimeric proteins of the invention can be cloned. A person with skill in the art understands that additional methods are available and are possible.

The invention also provides for constructs and/or vectors that comprise nucleotides that encode for said influenza M1, M2, peptides and/or chimeric proteins described above. Said vector can be, for example, a phage, plasmid, viral, or retroviral vector. The constructs and/or vectors that comprise the above constructs should be operatively linked to an appropriate promoter, such as the AcMNPV polyhedrin promoter (or other baculovirus), phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the protein to be translated.

Expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan. In one embodiment, said vector that comprises an influenza M1, an influenza M2 nucleotide, or portions thereof, and/or a chimeric protein described above is pFastBac

Next, the recombinant constructs mentioned above could be used to transfect, infect, or transform and can express influenza M1, M2, or portions thereof, and/or any chimeric protein described above into eukaryotic cells and/or prokaryotic cells. Thus, the invention provides for host cells that comprise a vector (or vectors) that contain nucleic acids comprising the above-described construct(s).

Among eukaryotic host cells are yeast, insect, avian, plant, C. elegans (or nematode) and mammalian host cells. Non-limiting examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K. lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Prokaryotic host cells include bacterial cells, for example, E. coli, B. subtilis, and mycobacteria.

The present invention comprises a method of producing said peptides, chimeric proteins and VLPs of the invention, comprising transfecting at least one vector encoding an influenza M1 protein and/or M2-M1 and/or M2-HA and/or M2-NA chimeric proteins.

Vectors, e.g., vectors comprising polynucleotides the above constructs, can be transfected into host cells according to methods well known in the art. For example, introducing nucleic acids into eukaryotic cells can be by calcium phosphate co-precipitation, electroporation, microinjection, lipofection, and transfection employing polyamine transfection reagents. In one embodiment, said vector is a recombinant baculovirus. In another embodiment, said recombinant baculovirus is transfected into a eukaryotic cell. In a preferred embodiment, said cell is an insect cell. In another embodiment, said insect cell is a Sf9 cell.

This invention also provides for constructs and methods that will increase the efficiency of peptide, chimeric protein and/or VLP production. For example, the addition of leader sequences to the constructs described above can improve the efficiency of protein transporting within the cell. For example, a heterologous signal sequence can be fused to peptides and/or chimeric proteins of the invention. In one embodiment, the signal sequence can be derived from the gene of an insect cell. In another embodiment, the signal peptide is the chitinase signal sequence, which works efficiently in baculovirus expression systems.

Methods to grow cells engineered to produce peptides and/or VLPs of the invention include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, said bioreactor is a stainless steel chamber. In another embodiment, said bioreactor is a pre-sterilized plastic bag (e.g. Cellbag®, Wave Biotech, Bridgewater, N.J.). In other embodiment, said pre-sterilized plastic bags are about 50 L to 1000 L bags.

Peptide (or chimeric protein) purification techniques are well known to those of skill in the art. These techniques involve, at one level, the crude fractionation of the cellular milieu to peptide and non-peptide fractions. Having separated the peptide from unwanted proteins, the peptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is reverse phase HPLC, followed by characterization of purified product by liquid chromatography/mass spectrometry (LC/MS) and Matrix-Assisted Laser Desorption Ionization (MALDI) mass spectrometry. Additional confirmation of purity is obtained by determining amino acid analysis.

Certain aspects of the present invention concern the purification, and in particular embodiments, the substantial purification, of an encoded chimeric protein or peptide. The term “purified peptide” as used herein, is intended to refer to a composition, isolatable from other components, wherein the peptide is purified to any degree relative to its naturally obtainable state. A purified peptide therefore also refers to a peptide, free from the environment in which it may naturally occur. Generally, “purified” will refer to a peptide composition that has been subjected to fractionation to remove various other components, and which composition substantially retains its expressed biological activity. In one embodiment, purified peptide will refer to a composition in which the peptide forms the major component of the composition, such as constituting about 50%, about 60%, about 70%, about 80%, about 90%, about 95% or more of the peptides in the purified sample.

Various techniques suitable for use in peptide purification will be well known to those of skill in the art. These include, for example, precipitation with ammonium sulphate, PEG, antibodies, and the like; heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the peptides always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed, utilizing an HPLC apparatus, will generally result in a greater “-fold” purification than the same technique utilizing a low pressure chromatography system.

In addition, it is contemplated that a combination of anion exchange and/or immunoaffinity chromatography may be employed to produce purified hybrid peptide compositions of the present invention.

VLP production, isolation and purification are also known in the art. VLPs should be isolated using methods that preserve the integrity thereof, such as by gradient centrifugation, e.g., cesium chloride, sucrose and iodixanol, as well as standard purification techniques including, e.g., ion exchange and gel filtration chromatography.

The following is an example of how VLPs of the invention can be made, isolated and purified. Usually VLPs are produced from recombinant cell lines engineered to create VLPs when said cells are grown in cell culture (see above). A person of skill in the art would understand that there are additional methods that can be utilized to make and purify VLPs of the invention, thus the invention is not limited to the method described.

Production of VLPs of the invention can start by seeding Sf9 cells (non-infected) into shaker flasks, allowing the cells to expand and scaling up as the cells grow and multiply (for example from a 125-ml flask to a 50 L Wave bag). The medium used to grow the cell is formulated for the appropriate cell line (preferably serum free media, e.g. insect medium ExCell-420, JRH). Next, said cells are infected with recombinant baculovirus at the most efficient multiplicity of infection (e.g. from about 1 to about 3 plaque forming units per cell).

Once infection has occurred, the avian M1 protein and/or any chimeric protein described above, are expressed from the virus genome, self assemble into VLPs and are secreted from the cells approximately 24 to 72 hours post infection. Usually, infection is most efficient when the cells are in mid-log phase of growth (4−8×10⁶ cells/m1), are at least about 90% viable, and are diluted to 1−4×10⁶ cells/ml with fresh insect medium prior to infection with a recombinant baculovirus.

VLPs of the invention can be harvested approximately 48 to 96 hours post infection, when the levels of VLPs in the cell culture medium are near the maximum but before extensive cell lysis. The Sf9 cell density and viability at the time of harvest can be about 0.5×10⁶ cells/ml to about 1.5×10⁶ cells/ml with at least 20% viability, as shown by dye exclusion assay. Next, the medium is removed and clarified. NaCl can be added to the medium to a concentration of about 0.4 to about 1.0 M, preferably to about 0.5 M, to avoid VLP aggregation. The removal of cell and cellular debris from the cell culture medium containing VLPs of the invention can be accomplished by tangential flow filtration (TFF) with a single use, pre-sterilized hollow fiber 0.5 or 1.00 μm filter cartridge or a similar device.

Next, VLPs in the clarified culture medium can be concentrated by ultrafiltration using a disposable, pre-sterilized 500,000 molecular weight cut off hollow fiber cartridge. The concentrated VLPs can be diafiltrated against 10 volumes pH 7.0 to 8.0 phosphate-buffered saline (PBS) containing 0.5 M NaCl to remove residual medium components.

The concentrated, diafiltered VLPs can be furthered purified on a 20% to 60% discontinuous sucrose gradient in pH 7.2 PBS buffer with 0.5 M NaCl by centrifugation at 6,500×g for 18 hours at about 4° C. to about 10° C. Usually VLPs will form a distinctive visible band between about 30% to about 40% sucrose or at the interface (in a 20% and 60% step gradient) that can be collected from the gradient and stored. This product can be diluted to comprise 200 mM of NaCl in preparation for the next step in the purification process. This product contains VLPs and may contain intact baculovirus particles.

Further purification of VLPs can be achieved by anion exchange chromatography, or 44% isopycnic sucrose cushion centrifugation. In anion exchange chromatography, the sample from the sucrose gradient (see above) is loaded into column containing a medium with an anion (e.g. Matrix Fractogel EMD TMAE) and eluded via a salt gradient (from about 0.2 M to about 1.0 M of NaCl) that can separate the VLP from other contaminates (e.g. baculovirus and DNA/RNA). In the sucrose cushion method, the sample comprising the VLPs is added to a 44% sucrose cushion and centrifuged for about 18 hours at 30,000 g. VLPs form a band at the top of 44% sucrose, while baculovirus precipitates at the bottom and other contaminating proteins stay in the 0% sucrose layer at the top. The VLP peak or band is collected.

The intact baculovirus can be inactivated, if desired. Inactivation can be accomplished by chemical methods, for example, formalin or β-propiolactone (BPL). Removal and/or inactivation of intact baculovirus can also be largely accomplished by using selective precipitation and chromatographic methods known in the art, as exemplified above. Methods of inactivation comprise incubating the sample containing the VLPs in 0.2% of BPL for 3 hours at about 25° C. to about 27° C. The baculovirus can also be inactivated by incubating the sample containing the VLPs at 0.05% BPL at 4° C. for 3 days, then at 37° C. for one hour.

After the inactivation/removal step, the product comprising VLPs can be run through another diafiltration step to remove any reagent from the inactivation step and/or any residual sucrose, and to place the VLPs into the desired buffer (e.g. PBS). The solution comprising VLPs can be sterilized by methods known in the art (e.g. sterile filtration) and stored in the refrigerator or freezer.

The above techniques can be practiced across a variety of scales. For example, T-flasks, shake-flasks, spinner bottles, up to industrial sized bioreactors. The bioreactors can comprise either a stainless steel tank or a pre-sterilized plastic bag (for example, the system sold by Wave Biotech, Bridgewater, N.J.). A person with skill in the art will know what is most desirable for their purposes.

Expansion and production of baculovirus expression vectors and infection of cells with recombinant baculovirus to produce peptides, chimeric proteins and/or VLPs of the invention can be accomplished in insect cells, for example Sf9 insect cells as previously described. In one embodiment, the cells are SF9 infected with recombinant baculovirus engineered to produce peptides, chimeric proteins and/or VLPs of the invention.

### Pharmaceuticals or Vaccine Formulations and Administration

The pharmaceutical compositions useful herein contain a pharmaceutically acceptable carrier, including any suitable diluent or excipient, which includes any pharmaceutical agent that does not itself induce the production of an immune response harmful to the subject receiving the composition, and which may be administered without undue toxicity and peptide (including chimeric proteins described above) and/or VLPs of the invention. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic compositions for inducing a protective immune response in a subject. In another embodiment, said protective immune response is against an influenza virus comprising a HA selected from the group consisting of H1, H2, H3, H4, H5, H6, H7, H8, H9, H10, H11, H12, H13, H14, H15 and H16 and a NA is selected from the group consisting of N1, N2, N3, N4, N5, N6, N7, N8, and N9.

The invention includes two small conserved N terminus peptide sequences that were found in both seasonal and avian influenza viral isolates, MSLLTEVET and MSLLTEVETP, including peptides comprising N and/or C terminal extensions of about 1 to 10 amino acids. These variable length peptides may be used in any composition or method as described herein. Thus, the invention comprises at least two different types of formulations comprising the conserved N terminus M2 sequences. The peptide sequences MSLLTEVET and MSLLTEVETP are highly conserved amongst the avian influenza A and seasonal influenza A isolates.

Thus, in one embodiment the invention comprises an antigenic formulation comprising peptides and/or VLPs of the invention. In another embodiment, the invention comprises an antigenic formulation comprising a M2 peptide or fragments thereof. In another embodiment, said fragment comprises a peptide selected from the group consisting of MSLLTEVET, MSLLTEVETP, MSLLTEVETC and MSLLTEVETPC. In another embodiment, said fragment consists of a peptide selected from the group consisting of MSLLTEVET, MSLLTEVETP, MSLLTEVETC and MSLLTEVETPC. In a further embodiment, said antigenic formulation or formulated with an adjuvant. In another embodiment, said adjuvant are Novasomes. In another embodiment, the invention comprises an antigenic formulation comprising a M2-M1 chimeric protein or fragments thereof.

Furthermore, formulations for administration to a subject, in accordance with the invention, can comprise one or more peptides (peptides include chimeric proteins described above) of the invention. Accordingly, a peptide can be present in a formulation individually; alternatively, the peptide can exist as a homopolymer comprising multiple copies of the same peptide, or as a heteropolymer of various peptides. Polymers have the advantage of increased probability for immunological reaction and, where different peptide epitopes are used to make up the polymer, the ability to induce antibodies and/or T cells that react with different antigenic determinants of the antigen targeted for an immune response.

Peptides (including chimeric proteins), in most instances, should be associated with a carrier in order to increase the half-life of said peptide and/or chimeric proteins. Carriers that can be used with formulations of the invention (including vaccines) are well known in the art, and include, e.g. thyroglobulin, albumins such as human serum albumin, tetanus toxoid, polyamino acids such as poly L-lysine, poly L-glutamic acid, virus proteins, e.g. influenza, hepatitis B virus core protein, and the like. The formulations can contain a physiologically tolerable diluent such as water, or a saline solution, preferably phosphate buffered saline.

In another embodiment, said antigenic formulation comprise VLPs comprising an avian M1 and/or M2-M1 and/or M2-HA and/or M2-NA chimeric proteins.

In another embodiment, the invention comprises a vaccine comprising peptides and/or VLPs of the invention. In another embodiment, said vaccine comprising a M2 peptide or fragments thereof. In another embodiment, said fragment comprises a peptide selected from the group consisting of MSLLTEVET, MSLLTEVETP, MSLLTEVETC and MSLLTEVETPC. In another embodiment, said fragment consists of a peptide selected from the group consisting of MSLLTEVET, MSLLTEVETP, MSLLTEVETC and MSLLTEVETPC. In a further embodiment, said antigenic formulation or formulated with an adjuvant. In another embodiment said adjuvant are Novasomes.

In another embodiment, said vaccine comprise VLPs comprising an avian M1 and/or M2-M1 and/or M2-HA and/or M2-NA chimeric proteins, wherein the M2 protein may comprise a peptide as disclosed above.

Said formulations of the invention comprise a formulation comprising at least one peptide and/or chimeric protein and/or VLP of the invention and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The invention also provides for a pharmaceutical pack or kit comprising one or more containers filled with one or more of the ingredients of the formulations of the invention. In a preferred embodiment, the kit comprises two containers, one containing at least one peptide and/or chimeric protein and/or VLP of the invention and the other containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that at least one peptide and/or chimeric protein and/or VLP of the invention be packaged in a hermetically sealed container such as an ampoule or sachette indicating the quantity of composition. In one embodiment, a composition comprising at least one peptide and/or chimeric protein and/or VLP of the invention is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, a composition comprising at least one peptide and/or chimeric protein and/or VLP of the invention is supplied in liquid form in a hermetically sealed container indicating the quantity and concentration of said peptide, chimeric protein and/or VLP composition. Preferably, the liquid form of said composition is supplied in a hermetically sealed container at least about 50 μg/ml, more preferably at least about 100 μg/ml, at least about 200 μg/ml, at least 500 μg/ml, or at least 1 mg/ml of said peptides, chimeric proteins and/or VLPs of the invention.

Generally, peptides and/or chimeric proteins and/or VLPs of the invention of are administered in an effective amount or quantity sufficient to stimulate an immune response against one or more infectious agents. Preferably, administration of peptides and/or chimeric proteins and/or VLPs of the invention elicit immunity against influenza virus. Typically, the dose can be adjusted within this range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract. While any of the above routes of delivery results in an immune response, intranasal administration confers the added benefit of eliciting mucosal immunity at the site of entry of many viruses, including influenza.

Thus, the invention also comprises a method of formulating a vaccine or antigenic composition that induces immunity to an influenza infection, or at least one symptom thereof, to a subject, comprising adding to said formulation an effective dose of peptides and/or chimeric proteins and/or VLPs of the invention.

Methods of administering a composition comprising peptides and/or chimeric proteins and/or VLPs of the invention (vaccine and/or antigenic formulations) include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions of the present invention are administered intramuscularly, intravenously, subcutaneously, transdermally or intradermally. The compositions may be administered by any convenient route, for example, by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition comprising peptides and/or chimeric proteins and/or VLPs of the invention may induce an antibody or other immune response that is substantially higher than other routes of administration. In another embodiment, intranasal or other mucosal routes of administration of a composition comprising peptides and/or chimeric proteins and/or VLPs of the invention may induce an antibody or other immune response that will induce cross protection against all strains of influenza. Administration can be systemic or local.

In yet another embodiment, the vaccine and/or antigenic formulation is administered in such a manner as to target mucosal tissues in order to elicit an immune response at the site of immunization. For example, mucosal tissues such as gut associated lymphoid tissue (GALT) can be targeted for immunization by using oral administration of compositions that contain adjuvants with particular mucosal targeting properties. Additional mucosal tissues can also be targeted, such as nasopharyngeal lymphoid tissue (NALT) and bronchial-associated lymphoid tissue (BALT).

Vaccines and/or antigenic formulations of the invention may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose. In a preferred embodiment, a second dose is administered about one month after the first administration and a third dose is administered about six months after the first administration. In another embodiment, the second dose is administered about six months after the first administration. In another embodiment, said peptides and/or chimeric proteins and/or VLPs of the invention can be administered as part of a combination therapy. For example, peptides and/or chimeric proteins and/or VLPs of the invention of the invention can be formulated with other immunogenic compositions, antivirals and/or antibiotics.

The dosage of the pharmaceutical formulation can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. Said dosages can be determined from animal studies. A non-limiting list of animals used to study the efficacy of vaccines include the guinea pig, hamster, ferrets, chinchilla, mouse and cotton rat. Most animals are not natural hosts to infectious agents but can still serve in studies of various aspects of the disease. For example, any of the above animals can be dosed with a vaccine candidate, e.g. peptides and/or chimeric proteins and/or VLPs of the invention, to partially characterize the immune response induced, and/or to determine if any neutralizing antibodies have been produced. For example, many studies have been conducted in the mouse model because mice are small size and their low cost allows researchers to conduct studies on a larger scale. A preferred animal model for measuring vaccine efficacy that correlates to an effective response in humans is the ferret model.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

As also well known in the art, the immunogenicity of a particular composition can be enhanced by the use of non-specific stimulators of the immune response, known as adjuvants. Adjuvants have been used experimentally to promote a generalized increase in immunity against unknown antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this invention.

Exemplary adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MF-59, Novasomes®, MHC antigens may also be used.

In one embodiment of the invention, the adjuvant is a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiment, the vesicles are Novasomes. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928, herein incorporated by reference in their entireties for all purposes). In one embodiment, said M2, or fragments thereof, M2-M1 chimeric proteins and/or VLPs of the invention are formulated with Novasomes. In another embodiment, said fragments of M2 comprise the amino acid sequences, MSLLTEVET and/or MSLLTEVETP peptides. In another embodiment, said peptides are encapsulated in Novasomes. In another embodiment, said peptide is coupled to the surface of Novasomes. Peptides of the invention, including peptides comprising the amino acid sequences MSLLTEVET-C and MSLLTEVETP-C, can be coupled to the thiocholesterol on the surface of Novasomes. In another embodiment, administering said fragments prevents, ameliorates and/or treats influenza virus infection, wherein said influenza virus is an avian influenza virus. In another embodiment, said avian influenza virus is selected from the group consisting of H5N1, H9N2 and H7N7. In another embodiment, administering said fragments prevents or ameliorates influenza virus infection wherein said influenza virus is a seasonal influenza virus. In another embodiment, said seasonal or avian influenza virus comprises a HA selected from the group consisting of H1, H2, H3, H4, H5, H6, H7, H8, H9, H10, H11, H12, H13, H14, H15 and H16 and a NA selected from the group consisting of N1, N2, N3, N4, N5, N6, N7, N8, and N9.

Another method of inducing an immune response can be accomplished by formulating peptides and/or chimeric proteins and/or VLPs of the invention with “immune stimulators.” These are the body's own chemical messengers (cytokines) to increase the immune system's response. Immune stimulators include, but not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the peptides and/or chimeric proteins and/or VLPs of the invention, or can be administered separately. Either the protein or an expression vector encoding the protein can be administered to produce an immunostimulatory effect. Thus, in one embodiment, the invention comprises antigenic and vaccine formulations comprising an adjuvant and/or an immune stimulator.

Thus, one embodiment of the invention comprises a formulation comprising peptides and/or chimeric proteins and/or VLPs of the invention and adjuvant and/or an immune stimulator. In another embodiment, said adjuvant are Novasomes. In another embodiment, said formulation is suitable for human administration. In another embodiment, the formulation is administered to a subject orally, intradermally, intranasally, intramuscularly, intraperitoneally, intravenously or subcutaneously. In another embodiment, different peptides and/or chimeric proteins and/or VLPs of the invention are blended together to create a multivalent formulation.

While stimulation of immunity with a single dose is preferred, additional dosages can be administered, by the same or different route to achieve the desired effect. In neonates and infants, for example, multiple administrations may be required to elicit sufficient levels of immunity. Administration can continue at intervals throughout childhood, as necessary to maintain sufficient levels of protection against influenza infections (usually once a year). Similarly, adults who are particularly susceptible to repeated or serious infections, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

### Methods of Stimulating an Immune Response

The peptides and/or chimeric proteins and/or VLPs of the invention are useful for preparing compositions that stimulate an immune response that confers immunity to influenza viruses. Both mucosal and cellular immunity may contribute to immunity to influenza infection and disease. Peptides, chimeric proteins and/or VLPs of the invention can induce immunity in a subject (e.g., a human) when administered to said subject. The immunity results from an immune response against the peptides and/or chimeric proteins and/or VLPs of the invention that protects and/or ameliorates influenza infection and/or ameliorates at least one symptom thereof. In some instances, if the said subject is infected, said infection will be asymptomatic. The response may be not a fully protective response. In this case, if said subject is infected with an influenza virus, the subject will experience reduced symptoms or a shorter duration of symptoms compared to a non-immunized subject.

The invention also comprises a method of inducing immunity to influenza virus infection, or at least one symptom thereof in a subject, comprising administering a M2 peptide, or fragment thereof, chimeric protein and/or VLPs of the invention. In another embodiment, said induction of immunity reduces duration of influenza symptoms. In another embodiment, the invention includes a method to induce substantial immunity to influenza virus infection or at least one symptom thereof.

In one embodiment, the invention comprises a method of inducing immunity to an infection, or at least one symptom thereof, in a subject, comprising administering at least one effective dose of a formulation comprising peptides and/or chimeric proteins and/or VLPs of the invention. In another embodiment, the invention comprises a method of vaccinating a mammal against influenza comprising administering to said mammal a protection-inducing amount of a formulation comprising peptides and/or chimeric proteins and/or VLPs of the invention. In one embodiment, said method comprises administering a formulation comprising peptides and/or chimeric proteins and/or VLPs of the invention.

In another embodiment, the invention comprises a method of inducing a protective antibody response to an infection, or at least one symptom thereof in a subject, comprising administering at least one effective dose of a formulation comprising peptides and/or chimeric proteins and/or VLPs of the invention.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. Antibodies exist as intact immunoglobulins or as a number of well-characterized fragments produced by digestion with various peptidases.

In another embodiment, the invention comprises a method of inducing a protective cellular response to an infection or at least one symptom thereof in a subject, comprising administering at least one effective dose of a formulation comprising peptides and/or chimeric proteins and/or VLPs of the invention. Cell-mediated immunity also plays a role in recovery from infection and may prevent additional complication and contribute to long term immunity.

As mentioned above, said formulation comprising peptides and/or chimeric proteins and/or VLPs of the invention prevent or reduce at least one symptom of an infection in a subject when administered to said subject. Most symptoms of most infections are well known in the art. Thus, the method of the invention comprises the prevention or reduction of at least one symptom associated with an infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a influenza infection or additional symptoms, a reduced severity of a influenza symptoms or a suitable assays (e.g. antibody titer and/or T-cell activation assay). The objective assessment comprises both animal and human assessments.

This invention is further illustrated by the following examples that should not be construed as limiting. The contents of all references, patents and published patent applications cited throughout this application, as well as the Figures and the Sequence Listing, are incorporated herein by reference for all purposes.

### Example 1

### M2 Vaccine Formulated With Novasome Adjuvant

M2 protein from influenza A/Sydney/5/97 (H3N2) virus was expressed in Sf9 cells at high levels and reacted with the monoclonal antibody 14C2, which was raised from M2 protein. This M2 protein can be formulated with Novasomes for use as an influenza vaccine. In addition, influenza M2 from A/Philippines/2/82/BS (H3N2) protein is expressed in, and purified from, Sf9 cells. The purified M2 may also be formulated with Novasomes for the use as an influenza vaccine. The sequence of M2 for Influenza virus is shown, ACCESSION No. U08863.

(SEQ ID NO. 8)

MSLLTEVETPIRNEWGCRCNGSSDPLTIAANIIGILHLTLWILDRLFFKC
IYRRFKYGLKGGPSTEGVPKSMREEYRKEQQSAVDADDGHFVSIELE

M2 has demonstrable in vitro cellular cytoxicity that can be overcome by addition of amantidine to the tissue culture media.

These formulations are used to immunize animals and humans. Active and passive protection studies are completed in animal models. Rates of seasonal Influenza are monitored in immunized and non-immunized subjects to determine the efficacy of the vaccines in preventing cases of seasonal influenza.

### Example 2

### Identification of Smaller Conserved N Terminus Amino Acid Sequence

By aligning several M2 protein sequences two smaller conserved N terminus amino acid sequences found in both seasonal and avian Influenza isolates were identified. These are MSLLTEVET and MSLLTEVETP. The M2 Influenza A peptide sequences MSLLTEVET and MSLLTEVETP are highly conserved amongst the avian Influenza A isolates as illustrated on FIG. 4. A/Wild Duck/Nanchang/2-0480/2000(H9N2) and A/FPV/Dobson (H7N7), not shown, also have these conserved sequences.

Both conserved N terminus M2 peptides formulations are prepared with Novasomes by mixing said adjuvant with the amino acid sequences, MSLLTEVET, MSLLTEVETP or longer. Novasomes comprising MSLLTEVET, MSLLTEVETP or longer are encapsulated within the Novaysomes or are associated with Novaysomes via an electrostatic or other association. In another formulation the peptides MSLLTEVET-C, MSLLTEVETP-C or longer are coupled to thiocholesterol on the surface of Novasomes via the C terminus cysteine of said peptides.

### Example 3

### M2-M1 Chimeric Vaccine Construct

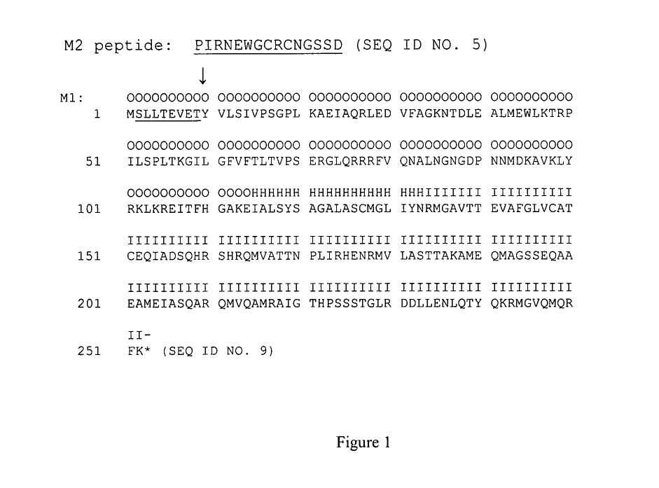

Peptides of 15 amino acid in length derived from M2 are inserted into the N-terminal part of M1 protein that contains the N-terminal portion of the M2 extracellular domain (as shown on FIG. 1 with the M2 polypeptide derived from A/Philippines and M1 protein derived from A/Fujian/411/02). The resulting chimeric M2-M1 protein has the complete M2 extracellular region (underlined). This chimeric protein can be formulated in a vaccine for administration to a subject or can be expressing in a host cell to form VLPs.

Virus-like particles (VLP) are formed with the resulting M2-M1 chimeric protein containing the complete M2 extracellular domain. VLPs are purified and used as an influenza vaccine, with or without Novasomes. The M2-M1 chimeric protein will likely have the complete M2 extracellular domain (˜24 amino acids) exposed on the surface of VLPs because predictions show the N-terminal parts of both M1 and M2 are exposed on the outer surface (see predictions below, FIGS. 2 and 3). M1 is also conserved among influenza A viruses (FIG. 5) and are important vaccine component.

In case the M2-M1 protein can have difficulty in forming VLPs due to steric hindrance due to the bulky M2 peptide insert, the M2-M1 chimeric protein may be used to assemble VLPs in the presence of the unmodified M1 protein. In this case, VLPs will be comprised of at least the two proteins: (1) M2-M1 chimeric protein, and (2) unmodified M1 protein.

### Example 4

### M2-HA or M2-NA Comprising VLP Vaccines

Virus-like particles (VLP) are formed when an influnza M1 protein is expressed in a host cell with either M2-HA and/or M2-NA chimeric construct expressed in said host cell. The VLP comprises the complete M2 extracellular domain expressed on the surface of the VLP. VLPs are purified and used as an influenza vaccine, with or without Novasomes. In this case, VLPs will be comprised of at least the two proteins: (1) M2-HA and/or M2 NA chimeric protein, and (2) unmodified M1 protein.

### Example 5

### Immunization of Mice with Functional Homotypic Recombinant Influenza VLPs

The immunogenicity of the recombinant influenza VLPs is ascertained by immunization of mice followed by western blot analysis of immune sera. Recombinant VLPs (e.g. 1 μg/injection) comprising M2-M1, M2-HA or M2-NA chimera proteins from avian influenza virus is purified on sucrose gradients. These VLPs are inoculated into mice at a specified immunization schedule. The mice are bled from the supraorbital cavity and tested for anti-influenza activity.

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method for the prevention, amelioration and/or treatment of influenza virus infection in a subject, comprising administering to said subject a M2 peptide or fragment thereof.

2. The method of claim 1, wherein said M2 peptide is formulated with an adjuvant.

3. The method of claim 2, wherein said adjuvant is Novasomes.

4. The method of claim 1, wherein said fragment comprises a peptide selected from the group consisting of MSLLTEVET (SEQ ID NO: 1), MSLLTEVETP (SEQ ID NO: 2), MSLLTEVETC (SEQ ID NO: 3) and MSLLTEVETPC (SEQ ID NO: 4).

5. The method of claim 1, wherein said fragment consists of a peptide from selected the group consisting of MSLLTEVET (SEQ ID NO: 1), MSLLTEVETP (SEQ ID NO: 2), MSLLTEVETC (SEQ ID NO: 3) and MSLLTEVETPC (SEQ ID NO: 4).

6. The method of claim 4, wherein said peptide is formulated with an adjuvant.

7. The method of claim 6, wherein said adjuvant is Novasomes.

8. The method of claim 4, wherein said peptide is coupled to the surface of Novasomes.

9. The method of claim 1, wherein said influenza virus is an avian influenza virus.

10. The method of claim 9, wherein said influenza virus is selected from the group consisting of H5H1, H9N2 and H7N7.

11. The method of claim 1, wherein said influenza virus is a seasonal influenza virus.

12. The method of claim 1, wherein said methods prevents, ameliorates, or treats influenza infections of more than one strain, clade and/or antigenic variation.

13. An antigenic formulation or vaccine comprising a M2 peptide or fragments thereof.

14.-20. (canceled)

21. A chimeric M2-M1 protein, wherein a M2 peptide fragment is fused at or near the N-terminus of the M1 protein.

22. The chimeric protein of claim 21, wherein said M2 peptide fragment consists of PIRNEWGCRCNGSSD (SEQ ID NO: 5).

23. The chimeric protein of claim 21, wherein said M2 peptide fragment is inserted between residues 9 and 10.

24. A chimeric M2-HA protein, wherein the transmembrane and/or C-terminal domain of influenza HA is fused to the external domain of M2.

25. A chimeric M2-NA protein, wherein the transmembrane and/or C-terminal domain of influenza NA is fused to the external domain of M2.

26. A virus-like particle (VLP) comprising the chimeric M2-M1 protein of claim 21.

27. The VLP of claim 26, wherein said VLP further comprises an intact M1 protein.

28. A VLP comprising the chimeric M2-HA protein of claim 24.

29. The VLP of claim 28, comprising an influenza M1 protein.

30. A VLP comprising the chimeric M2-NA protein of claim 25.

31. The VLP of claim 30, comprising an influenza M1 protein.

32.-34. (canceled)


SEQUENCE LISTING (19 sequences)

SEQ ID NO: 1 - protein, 9 aa, Influenza virus
MSLLTEVET

SEQ ID NO: 2 - protein, 10 aa, Influenza virus
MSLLTEVETP

SEQ ID NO: 3 - protein, 10 aa, Influenza virus
MSLLTEVETC

SEQ ID NO: 4 - protein, 11 aa, Influenza virus
MSLLTEVETPC

SEQ ID NO: 5 - protein, 15 aa, Influenza virus
PIRNEWGCRCNGSSD

SEQ ID NO: 6 - protein, 10 aa, Influenza virus
CMSLLTEVET

SEQ ID NO: 7 - protein, 11 aa, Influenza virus
CMSLLTEVETP

SEQ ID NO: 8 - protein, 97 aa, Influenza virus
MSLLTEVETPIRNEWGCRCNGSSDPLTIAANIIGILHLTLWILDRLFFKCIYRRFKYGLKGGPSTEGVPKSMREEYRKEQQSAVDADDGHFVSIELE

SEQ ID NO: 9 - protein, 252 aa, Influenza virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVATTNPLIRHENRMVLASTTAKAMEQMAGSSEQAAEAMEIASQARQMVQAMRAIGTHPSSSTGLRDDLLENLQTYQKRMGVQMQRFK

SEQ ID NO: 10 - protein, 97 aa, Influenza virus
MSLLTEVETPTRNEWECRCSDSSDPIVVAANIIGILHLILWILDRLFFKCIYRRLKYGLKRGPATAGVPESMREEYRQEQQSAVDVDDGHFVNIELE

SEQ ID NO: 11 - protein, 97 aa, Influenza virus
MSLLTEVETPTRNEWECRCSDSSDPLVVAASIIGILHLILWILDRLFFKCIYRRLKYGLKRGPSTAGVPESMREEYRQEQQSAVDVDDGHFVNIELE

SEQ ID NO: 12 - protein, 23 aa, Influenza virus
MSLLTEVETPTRNEWECRCSDSS

SEQ ID NO: 13 - protein, 97 aa, Influenza virus
MSLLTEVETLTRNGWECKCSDSSDPLVVAASIIGILHLILWILDRLFFKCIYRRFKYGLKRGPSTEGVPESMREEYRQEQQNAVDVDDGHFVNIELE

SEQ ID NO: 14 - protein, 97 aa, Influenza virus
MSLLTEVETPTRNGWECRCSDSSDPLVIAASIIGILHLILWILDRLFVKCIYRRLKYGLKRGPSTEGVLESMREEYRQEQQNAVDVDDGHFVNIELE

SEQ ID NO: 15 - protein, 252 aa, Influenza virus; identical to SEQ ID NO: 9

SEQ ID NO: 16 - protein, 252 aa, Influenza virus
MSLLTEVETYVLSIIPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEIALSYSAGALASCMGLIYNRMGAVTTESAFGLICATCEQIADSQHKSHRQMVTTTNPLIRHENRMVLASTTAKAMEQMAGSSEQAAEAMEVASQARQMVQAMRAIGTHPSSSTGLKNDLLENLQAYQKRMGVQMQRFK

SEQ ID NO: 17 - protein, 252 aa, Influenza virus
MSLLTEVETYVLSIIPSGPLKAEIAQKLEDVFAGKNTDLEALMEWLKTRPILSPLTKGILGFVFTLTVPSERGLQRRRFVQNALNGNGDPNNMDRAVKLYKKLKREITFHGAKEVSLSYSTGALASCMGLIYNRMGTVTTEVAFGLVCATCEQIADSQHRSHRQMATITNPLIRHENRMVLASTTAKAMEQMAGSSEQAAEAMEVANQARQMVQAMRTIGTHPNSSAGLRDNLLENLQAYQKRMGVQMQRFK

SEQ ID NO: 18 - protein, 252 aa, Influenza virus
MSLLTEVETYVLSIIPSGPLKAEIAQKLEDVFAGKNTDLEALMEWLKTRPILSPLTKGILGFVFTLTVPSERGLQRRRFVQNALNGNGDPNNMDRAVKLYKKLKREITFHGAKEVALSYSTGALASCMGLIYNRMGTVTTEVAFGLVCATCEQIADSQHRSHRQMATITNPLIRHENRMVLASTTAKAMEQMAGSSEQAAEAMEIANQARQMVQAMRTIGTHPNSSAGLRDNLLENLQAYQKRMGVQMQRFK

SEQ ID NO: 19 - protein, 252 aa, Influenza virus
MSLLTEVETYVLSIIPSGPLKAEIAQRLEDVFAGKNTDLEALMEWLKTRPILSPLTKGILGFVFTLTVPSERGLQRRRFVQNALNGNGDPNNMDRAVKLYKKLKREMTFHGAKEVALSYSTGALASCMGLIYNRMGTVTTEVALGLVCATCEQIADAQHRSHRQMATTTNPLIRHENRMVLASTTAKAMEQMAGSSEQAAEAMEVASQARQMVQAMRTIGTHPSSSAGLKDDLIENLQAYQKRMGVQMQRFK